(19) United States
(12) Patent Application Publication
(10) Pub. No.: US 2023/0191384 A1
(43) Pub. Date: Jun. 22, 2023

(54) Pt-thixantphos-iodine complex and Pt-thixantphos-bromine complex

(71) Applicant: EVONIK OPERATIONS GMBH, Essen (DE)

(72) Inventors: Carolin Schneider, Monheim am Rhein (DE); Ralf Jackstell, Rostock (DE); Matthias Beller, Ostseebad Nienhagen (DE); Robert Franke, Marl (DE)

(73) Assignee: EVONIK OPERATIONS GMBH, Essen (DE)

(21) Appl. No.: 18/064,958

(22) Filed: Dec. 13, 2022

(30) Foreign Application Priority Data
Dec. 17, 2021 (EP) 21215344.9

(51) Int. Cl. B01J 31/24; B01J 31/30; C07F 15/00; C07C 45/50

(52) U.S. Cl. CPC B01J 31/2457; B01J 31/30; C07F 15/0086; C07C 45/505; B01J 2231/321

(57) ABSTRACT

Pt-thixantphos-iodine complex and Pt-thixantphos-bromine complex, and the use thereof for catalysis of a hydroformylation reaction.

The present invention relates to a Pt-thixantphos-iodine complex and Pt-thixantphos-bromine complex, and the use thereof for catalysis of a hydroformylation reaction.

P. Meessen et al., Journal of Organometallic Chemistry, 551, (1998), 165-170 describes the use of thixantphosPtCl₂ for the hydroformylation of methyl 3-pentenoate.

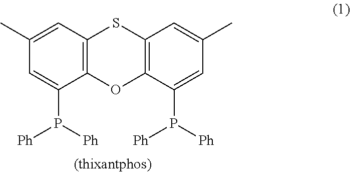

The problem addressed by the present invention is that of providing a novel complex. In the case of catalysis of hydroformylation reactions, the complex should afford a good yield.

This object is achieved by a complex according to claim 1.

Complex comprising:

a) Pt;

b) a ligand conforming to the formula (I):

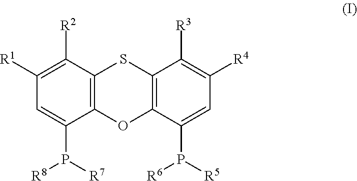

where R¹, R², R³, R⁴, R⁵, R⁶, R⁷, R⁸ are selected from: —H, —(C₁-C₁₂)-alkyl, —(C₆-C₂₀)-aryl;

c) an iodine ligand or bromine ligand.

The expression (C₁-C₁₂)-alkyl encompasses straight-chain and branched alkyl groups having 1 to 12 carbon atoms. These are preferably (C₁-C₈)-alkyl groups, more preferably (C₁-C₆)-alkyl, most preferably (C₁-C₄)-alkyl.

Suitable (C₁-C₁₂)-alkyl groups are especially methyl, ethyl, propyl, isopropyl, n-butyl, isobutyl, sec-butyl, tert-butyl, n-pentyl, 2-pentyl, 2-methylbutyl, 3-methylbutyl, 1,2-dimethylpropyl, 1,1-dimethylpropyl, 2,2-dimethylpropyl, 1-ethylpropyl, n-hexyl, 2-hexyl, 2-methylpentyl, 3-methylpentyl, 4-methylpentyl, 1,1-dimethylbutyl, 1,2-dimethylbutyl, 2,2-dimethylbutyl, 1,3-dimethylbutyl, 2,3-dimethylbutyl, 3,3-dimethylbutyl, 1,1,2-trimethylpropyl, 1,2,2-trimethylpropyl, 1-ethylbutyl, 1-ethyl-2-methylpropyl, n-heptyl, 2-heptyl, 3-heptyl, 2-ethylpentyl, 1-propylbutyl, n-octyl, 2-ethylhexyl, 2-propylheptyl, nonyl, decyl.

The expression (C₆-C₂₀)-aryl encompasses mono- or polycyclic aromatic hydrocarbyl radicals having 6 to 20 carbon atoms. These are preferably (C₆-C₁₄)-aryl, more preferably (C₆-C₁₀)-aryl.

Suitable (C₆-C₂₀)-aryl groups are especially phenyl, naphthyl, indenyl, fluorenyl, anthracenyl, phenanthrenyl, naphthacenyl, chrysenyl, pyrenyl, coronenyl. Preferred (C₆-C₂₀)-aryl groups are phenyl, naphthyl and anthracenyl.

In one embodiment, R⁵, R⁶, R⁷, R⁸ are —(C₆-C₂₀)-aryl.

In one embodiment, R⁵, R⁶, R⁷, R⁸ are —Ph.

In one embodiment, R¹ and R⁴ are —(C₁-C₁₂)-alkyl.

In one embodiment, R¹ and R⁴ are —CH₃.

In one embodiment, R² and R³ are —H.

In one embodiment, the compound of formula (I) has the structure (1):

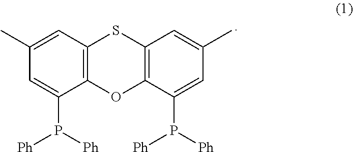

In one embodiment, the complex has exactly one ligand corresponding to formula (I).

In one embodiment, the complex has at least two iodine ligands.

In one embodiment, the complex has exactly two iodine ligands.

In one embodiment, the complex has at least two bromine ligands.

In one embodiment, the complex has exactly two bromine ligands.

As well as the complex per se, the use thereof for catalysis of a hydroformylation reaction is also claimed.

Use of a complex as described above for catalysis of a hydroformylation reaction.

The invention shall be elucidated in more detail hereinbelow with reference to working examples.

### EXPERIMENTAL DESCRIPTION

A vial was charged with PtX₂ (X=halogen), ligand, and an oven-dried stirrer bar. The vial is then sealed with a septum (PTFE-coated styrene-butadiene rubber) and phenolic resin cap. The vial is evacuated and refilled with argon three times. Toluene and 1-octene were added to the vial using a syringe. The vial was placed in an alloy plate, which was transferred to an autoclave of the 4560 series from Parr Instruments under an argon atmosphere. After purging the autoclave three times with CO/H₂, the synthesis gas pressure was increased to 40 bar at room temperature. The reaction was conducted at 80° C. for 18 h. On termination of the reaction, the autoclave was cooled to room temperature and cautiously decompressed. Yield and selectivity were determined by GC analysis.

Hydroformylation of 1-octene

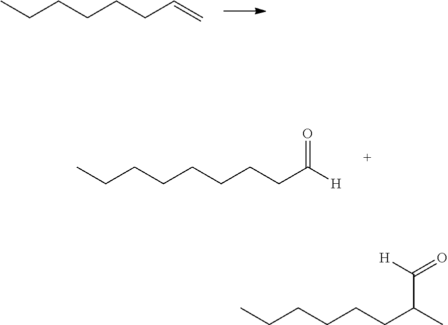

Reaction conditions:

1.0 mmol of 1-octene, 0.5 mol % of PtI₂, 2.0 equivalents of ligand, solvent: toluene, p(CO/H₂): 40 bar, T: 80° C., t: 18 h.

| Ligand | | Yield (%) |
|---|---|---|
| 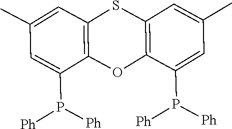 | (1)* | PtI₂*: 99 |

*process according to the invention

Variation of the Halogen

Reaction conditions:

10 mmol of 1-octene, 0.1 mol % of PtX₂, 2.2 equivalents of ligand (1), solvent: toluene, p(CO/H₂): 40 bar, T: 80° C., t: 20 h.

Yields:

PtI₂: 95%

PtBr₂: 48%

PtCl₂: <1%

As the experimental results show, the object is achieved by the complex according to the invention.

1. Complex comprising:
a) Pt;
b) a ligand conforming to the formula (I): 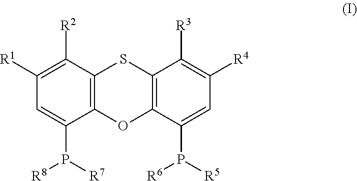
where R¹, R², R³, R⁴, R⁵, R⁶, R⁷, R⁸ are selected from: —H, —(C₁-C₁₂)-alkyl, —(C₆-C₂₀)-aryl;
c) an iodine ligand or bromine ligand.

2. Complex according to claim 1,
where R⁵, R⁶, R⁷, R⁸ are —(C₆-C₂₀)-aryl.

3. Complex according to claim 1,
where R⁵, R⁶, R⁷, R⁸ are —Ph.

4. Complex according to claim 1,
where R¹ and R⁴ are —(C₁-C₁₂)-alkyl.

5. Complex according to claim 1,
where R² and R³ are —H.

6. Complex according to claim 1,
wherein the compound of formula (I) has the structure (1):

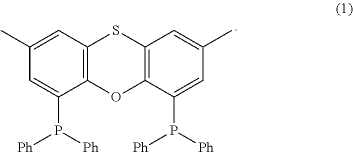

7. Complex according to claim 1,
wherein the complex has exactly one ligand conforming to the formula (I).

8. Complex according to claim 1,
wherein the complex has at least two iodine ligands.

9. Complex according to claim 8,
wherein the complex has exactly two iodine ligands.

10. Complex according to claim 1,
wherein the complex has at least two bromine ligands.

11. Complex according to claim 10,
wherein the complex has exactly two bromine ligands.

12. Use of a complex according to claim 1 for catalysis of a hydroformylation reaction.
